(19) United States
(12) Patent Application Publication
(10) Pub. No.: US 2024/0033315 A1
(43) Pub. Date: Feb. 1, 2024

(54) COMPOSITION HAVING FUNCTIONS OF MOISTURIZING, REPAIRING AND WHITENING AND USE THEREOF

(71) Applicant: INFINITUS (CHINA) COMPANY LTD., Jiangmen (CN)

(72) Inventors: Kaiye Yang, Jiangmen (CN); Guangrong Liu, Jiangmen (CN); Meiling Tai, Jiangmen (CN); Jiawen Chen, Jiangmen (CN); Shuang Gao, Jiangmen (CN); Lusheng Wang, Jiangmen (CN); Zhenlin Hu, Jiangmen (CN)

(21) Appl. No.: 18/070,723

(22) Filed: Nov. 29, 2022

(30) Foreign Application Priority Data
Jul. 29, 2022 (CN) 202210910283.8

(51) Int. Cl. A61K 36/8984; A61K 31/715; A61K 36/185; A61K 36/484; A61Q 19/02; A61Q 19/00; A61K 8/73; A61K 8/9794; A61K 8/9789; A61P 39/06; A61P 29/00

(52) U.S. Cl. CPC A61K 36/8984; A61K 31/715; A61K 36/185; A61K 36/484; A61Q 19/02; A61Q 19/007; A61K 8/73; A61K 8/9794; A61K 8/9789; A61P 39/06; A61P 29/00; A61K 2800/522; A61K 2800/5922

(57) ABSTRACT

Disclosed is a composition having functions of moisturizing, repairing and whitening and use thereof. The composition comprises Dendrobium candidum polysaccharide, a lotus extract and a Glycyrrhiza glabra extract. The lotus extract and the Glycyrrhiza glabra extract can inhibit pigmentation by inhibiting synthesis of melanin, inhibiting transfer of melanin globule, resisting oxidation and improving skin microcirculation, so as to exert the effect of special whitening. Through the synergistic synergy of the three components, and the compounded composition can reduce synthesis of melanin of skin's melanophores, promote expression of keratinocyte cell hydration-related factors, inhibit expression of inflammation-related factors, promote the ability of lipid synthesis and exert multiple effects of whitening, enhancement in moisturizing of the skin, alleviation in inflammation and discomfort of the skin, and improvement in barrier function of epidermis. In addition, the compounded composition may be applied to preparation of skin care products or skin formulation.

### CROSS-REFERENCE TO RELATED APPLICATION

The present disclosure is based on and claims the priority of the Chinese Patent Application No. 202210910283.8, filed on Jul. 29, 2022, the entire contents of which are incorporated herein by reference.

### TECHNICAL FIELD

The present disclosure belongs to the technical field of daily cosmetics, and particularly relates to a composition having functions of moisturizing, repairing and whitening and use thereof.

### BACKGROUND

As the largest body surface organ of a human body, skin can synthesize pigments to effectively prevent ultraviolet radiation in addition to providing an efficient physical barrier to prevent pathogenic microorganisms from entering or preventing bodily fluids from flowing out. In skin tissues, there are four main chromophores, including brown melanin, red oxygen and heme, blue reductive heme and yellow carotene and bile pigment, where a skin color and the ultraviolet ray sensitivity mainly depend on the content and distribution of the melanin. Excessive melanin may cause uneven skin pigmentation and dark skin color, and induce freckles, chloasma, age spots, even melanoma, and other pigment-related skin diseases, thereby affecting people's psychological and social activities to a certain extent. Therefore, in order to meet people's pursuit of skin aesthetics, numerous skin care companies and drug research institutes have invested a lot of researches in whitening and skin caring to solve the problem of abnormal skin pigment.

There are many ways to whiten skin. In addition to inhibiting melanin related enzyme activities, the ways to the whiten skin further include: by inhibiting transfer and distribution of melanosomes, accelerating degradation of the melanin, promoting updating of epidermis, resisting inflammation and oxidation and other mechanisms, production of the melanin is controlled or the quantity of active melanophores is reduced. However, skin whitening has potential hazards. The most important biological activity of the melanin as a polymer biological pigment is to prevent optical damages caused by ultraviolet rays by absorbing and scattering the ultraviolet rays. Ultraviolet irradiation not only can directly lead to biological damage to a certain extent, but also can indirectly cause destruction of DNA damages and repair mechanisms in a body, inhibition of a signal transduction pathway, degradation of an extracellular matrix and the like by producing active oxygen (ROS) such as superoxide and hydrogen peroxide. Under the normal physiological conditions, low-level ROS plays a positive role in cell signal transduction and regulation processes of cell activation, differentiation and growth. However, more and more researches prove that if the production of ROS goes beyond the antioxidant defense capacity of the body, it can result in unbalanced body oxidation resistance, thereby forming oxidative stress, promoting peroxidation of cytomembrane lipid components, changing structures and functions of an enzyme system, and then promoting oxidation of carbohydrates. When the activity of the enzyme changes, for example, activation of cysteinyl aspartate specific proteinase 3 (Caspase3) may induce apoptosis of cells, stimulate the cells to synthesize platelet activating factors and trigger aggregation of platelets and neutrophils, thereby causing and promoting an inflammatory response. The inflammatory response is a protective reaction that when body tissues are damaged or certain pathogenic microorganisms invade, an immune system is activated, inflammatory cells are infiltrated, and many cell factors are secreted. When the ROS reacts with an acyl double bond of an unsaturated fatty acid, peroxidation of lipid may be caused. Peroxidation of the lipid may further lead to improvement in permeability of a cell membrane or a mitochondrial membrane and promote aggravation of oxidative stress damages to cells or adjacent cells, so as to further promote the inflammatory response. Therefore, the whitening products on the market bring a strong whitening effect, which can often cause damages to the normal physiological function of the skin to a certain extent, and most of the whitening products only focus on the whitening effect, but ignore the side effect that the whitening ingredients can further cause dry skin. The dry skin is mainly manifested as rough and tight skin, and may further aggravate the skin sensitivity while bring discomfort, so that the human skin is more vulnerable to external damage. Therefore, it is urgent to reduce the side effects brought by whitening while focusing on whitening benefits.

The moisturizing structure of epidermal stratum corneum and water supply capacity of the dermis are important factors affecting the dry skin. The principle for skin moisturizing includes: oil moisturizing for preventing evaporation of water, hygroscopic moisturizing for absorbing external moisture, hydrating and moisturizing combined with hydration, and repairing and moisturizing for repairing keratinocytes. For oil moisturizing, oil may adhere to the skin for a long time to form a moisturizing barrier on the surface of the skin to reduce water loss, and lipids between stratum corneum cells are covalently bound by free sterols, free fatty acids and ceramides to form a hydrophobic lipid bilayer semi-permeable membrane which wraps around the stratum corneum cells and is filled between the stratum corneum cells, thereby effectively preventing passage of water.

Polysaccharide can form a hydrophilic and breathable protective membrane on the epidermis of the skin so as to reduce loss of percutaneous water. Meanwhile, the hydrophilicity of the polysaccharide contributes to absorption of water in the skin to increase the hydration of the stratum corneum. In addition, saussurea polysaccharide may reinforce the barrier function of the skin, enhance the hydration of the skin and increase the moisturizing function of the skin by inhibiting the activity of epidermal growth factor receptor (EGFR) and extracellular regulated protein kinases (ERK) and up-regulating the expression quantity and the activity of aquaporin 3 (AQP3).

The polysaccharide and the Glycyrrhiza glabra extract can down-regulate production of the ROS induced by ultraviolet rays (UVB), and reduce the degree of skin oxidative stress. The polysaccharide has good oxidation resistance. When low-dose UVB induces production of the ROS or generation of oxidative stress, the polysaccharide may activate a nuclear factor E2 related factor 2 (Nrf2) pathway, improve the activities of antioxidant enzymes such as superoxide dismutase (SOD) and glutathione peroxide (GSH-PX), reduce the level of the ROS, and alleviate oxidative stress damages. In addition, by mediating p53 signal pathway, the expression level of p53, p21 and Bax genes of apoptotic proteins is reduced, the expression level of the bimicrosomal gene 2 and the telomerase gene is increased, and the oxidative stress response of the body is inhibited. The Glycyrrhiza glabra may achieve oxidation resistance by capturing and neutralizing free radicals, and may also achieve balance regulation of oxidative stress and oxidation resistance by acting on cell signaling pathways, activating in-vivo transcription factors, and initiating the expression level of downstream protective genes, so as to alleviate the degree of skin oxidative stress.

The polysaccharide and the Glycyrrhiza glabra extract can down-regulate the expression level of proinflammatory cytokine interleukin-1α (IL-1α), interleukin-1β (IL-1β), interleukin-6 (IL-6) and tumor necrosis factor α (TNF-α), and alleviate the skin sensitivity and the inflammatory response. The Glycyrrhiza glabra extract may down-regulate the expression level of inflammatory factors to alleviate inflammatory response through the following three ways: (1) reducing the production of NO; (2) inhibiting the activation of cyclic guanosine phosphate (cGMP); (3) activating a large conductance calcium activated potassium channel (BK-Ca²⁺). By reducing the activation level of phosphatidylinositol 3 kinase/protein kinase B (PI3K/Akt) pathway, the lotus extract may, reduce the expression level of downstream inflammation related genes, inhibit the production of inflammatory factors IL-6, IL-1β and IL-10 and then alleviate the inflammatory response.

By increasing the mRNA expression levels of lipid synthesis related factor elongase of very long chain fatty acids (ELOVL1, ELOVL4 and ELOVL6), ceramide synthases (CerS3, CerS4), fatty acid synthase (FAS) and serine palmitoyl transferase (SPT), the polysaccharide can promote lipid production and enhance the skin moisturizing and barrier functions.

The melanin is considered as a protective pigment against light damages caused by the ultraviolet rays, but it self also has certain toxicity. Excessive accumulation of the melanin in the skin can lead to pigment related skin diseases such as dark complexion and freckles, and even skin cancer. Aiming to the problem of melanin deposition, many whitening products on the market at present achieve the whitening effect by inhibiting the activity of melanin related enzymes and other methods, but the effect is not ideal. Most of the whitening products may reduce skin hydration while achieving the whitening effect and then cause the dry skin and other problems to a certain extent. However, at present, many skin moisturizing products developed in the market mainly focus on moisturizing. They mainly achieve the skin moisturizing effect by preventing internal moisture of the skin from emanating, promoting the skin to absorb external moisture and other methods, and do not have obvious whitening effect. Therefore, developing a whitening product with multiple effects such as moisturizing is of great significance to the current skin care product market.

The existing researches show that the polysaccharide may reduce loss of the percutaneous water, promote absorption of the water in skin and increase hydration of the stratum corneum, thereby playing a full role in moisturizing. The Glycyrrhiza glabra extract has the ability of resisting oxidation and inhibiting the activity of tyrosinase. The lotus extract may also inhibit formation of the melanin by weakening the activity of the tyrosinase so as to realize the whitening effect. In this research, it is found for the first time that a polysaccharide-whitening factor complex can exert multiple effects of water replenishing, moisturizing, oxidative stress prevention, alleviation in skin inflammatory reaction and strengthening in skin barrier on the basis of whitening, may achieve both whitening and moisturizing, and effectively makes up for the deficiency of the dry skin caused by the whitening products on the market.

### SUMMARY

The objective of the first aspect of the present disclosure is to provide a composition.

The objective of the second aspect of the present disclosure is to provide use of the composition according to the first aspect of the present disclosure.

The objective of the third aspect of the present disclosure is to provide a product.

The technical solution used in the present disclosure is as follows:

In the first aspect of the present disclosure, provided is a composition, comprising Dendrobium candidum polysaccharide, a lotus extract and a Glycyrrhiza glabra extract.

Preferably, a mass ratio of the Dendrobium candidum polysaccharide to the lotus extract to the Glycyrrhiza glabra extract is (1-5):(1-3):(1-3).

Preferably, the mass ratio of the Dendrobium candidum polysaccharide to the lotus extract to the Glycyrrhiza glabra extract is (2-5):(1-3):(1-2).

Preferably, the mass ratio of the Dendrobium candidum polysaccharide to the lotus extract to the Glycyrrhiza glabra extract is (1-3):(1-3):(1-3).

Preferably, the mass ratio of the Dendrobium candidum polysaccharide to the lotus extract to the Glycyrrhiza glabra extract is (2-3):(1-3):(1-3).

Preferably, the mass ratio of the Dendrobium candidum polysaccharide to the lotus extract to the Glycyrrhiza glabra extract is 3:(1-3):(1-3).

Preferably, the mass ratio of the Dendrobium candidum polysaccharide to the lotus extract to the Glycyrrhiza glabra extract is 3: (2-3):(2-3).

Preferably, the mass ratio of the Dendrobium candidum polysaccharide to the lotus extract to the Glycyrrhiza glabra extract is 3:(1-2):(1-2).

Preferably, the mass ratio of the Dendrobium candidum polysaccharide to the lotus extract to the Glycyrrhiza glabra extract is 3: (2-3):(2-3).

Preferably, the Dendrobium candidum polysaccharide, the lotus extract and the Glycyrrhiza glabra extract may be commercially available, or may be extracted from Dendrobium candidum, lotus and Glycyrrhiza glabra as raw materials respectively.

Preferably, a method for preparing the Dendrobium candidum polysaccharide comprises:
(1) leaching Dendrobium candidum with water, and performing solid-liquid separation to obtain a filtrate;
(2) performing concentration and alcohol precipitation on the filtrate, and performing solid-liquid separation to collect precipitates; and
(3) dissolving the precipitates into water to obtain a polysaccharide solution, removing free proteins, and decoloring, purifying and drying to obtain the Dendrobium candidum polysaccharide.

Preferably, in step (1), the leaching is performed for 1-3 times, and each leaching lasts for 1-2.5 h.

Preferably, in step (1), a material-to-liquid ratio of the Dendrobium candidum to the water is 1 g:(10-40) mL.

Preferably, in step (1), a method for performing the solid-liquid separation is filtration, preferably, 80-120 mesh filtration.

Preferably, in step (2), the condition of concentration is as follows: the filtrate is concentered to 1/10-⅕ at 70-80□.

Preferably, in step (2), a concentration of alcohol in alcohol precipitation is 70-90%, and is the alcohol precipitation lasts for 1-3 h.

Preferably, in step (2), a method for performing the solid-liquid separation is filtration, preferably, 250-350 mesh filtration.

Preferably, in step (3), a concentration of the solution formed after dissolving the precipitates into the water is 0.1-1%.

Preferably, in step (3), specifically, the free proteins are removed by using savage reagent.

Preferably, a volume ratio of the polysaccharide solution to the savage reagent is (4-6):1.

Preferably, in the savage reagent, a volume ratio of chloroform:n-butyl alcohol is (3-5):1 (V/V).

Preferably, the decoloring method comprises: performing adsorption with macroporous resin.

Preferably, the macroporous resin comprises macroporous resin AB-8.

Preferably, the decoloring method specifically comprises: after performing adsorption with the macroporous resin, removing a washing solution, adding a polysaccharide solution for mixing, and then performing concentration to prepare polysaccharide lyophilized powder.

Preferably, the mixing lasts for 2-5 h.

Preferably, the condition of concentration is as follows: the mixing solution is concentered to ⅛- 1/12 at 60-70□.

Preferably, the lyophilized powder is prepared by freeze drying.

Preferably, the condition of freeze drying is as follows: freeze drying is performed for 44-52 h at −70 to −90□.

Preferably, in step (3), purifying specifically comprises: dissolving the polysaccharide lyophilized powder into water for centrifugation, taking a supernatant, separating the supernatant via a chromatographic column, and then performing concentration and drying.

Preferably, a material-to-liquid ratio of the polysaccharide lyophilized powder to the water is (15-25) mg: 1 mL.

Preferably, the condition of centrifugation is as follows: centrifugation is performed for 8-12 min at 4500-7500 r/min.

Preferably, the chromatographic column includes a gel chromatographic column, preferably, a G100 sephadex gel chromatographic column.

Preferably, the condition for concentration is as follows: the separated supernatant is concentered to ⅛- 1/12 at 60-70□.

Preferably, the drying comprises freeze drying.

Preferably, the condition for freeze drying is as follows: freeze drying is performed for 44-52 h at −70 to −90□.

Preferably, a molecular weight of the Dendrobium polysaccharide is 0.3-100 kDa.

Preferably, the molecular weight of the Dendrobium polysaccharide is 0.3-27 kDa.

In the second aspect of the present disclosure, use of the composition according to the first aspect of the present disclosure in preparation of a product is provided.

Preferably, the product has at least one function of (a)-(j):
(a) reducing a content of melanin;
(b) promoting a expression level of aquaporin AQP3;
(c) inhibiting production of ROS;
(d) inhibiting expression of inflammatory factors;
(e) promoting expression of lipid synthesis-related factors;
(f) promoting lipid synthesis;
(g) whitening;
(h) moisturizing;
(i) resisting oxidation; and
(j) resisting inflammation.

Preferably, the product is a cosmetic or a drug.

In the third aspect of the present disclosure, provided is a product, comprising the composition according to the first aspect of the present disclosure.

Preferably, the product is a cosmetic or a drug.

Preferably, the cosmetic is a skin cream, a lotion, an essence, skin care gel, a lotion or a facial mask.

Preferably, a dosage form of the cosmetic comprises: powder, paste, gel, suspension, fluid, liquid, solid, semi-solid, solid gel or semi-solid gel.

Preferably, the cosmetic further comprises an excipient, wherein the excipient is at least one selected from the group consisting of a preservative, a chelating agent, an emollient, a flavor, a moisturizer, a colorant, an emulsifier, an antioxidant and a skin conditioner.

Preferably, the drug comprises a topical formulation.

The present disclosure has the beneficial effects:

The present disclosure provides a polysaccharide-whitening factor complex comprising Dendrobium candidum polysaccharide, a lotus extract and a Glycyrrhiza glabra extract. Different from the acting mechanism of the traditional whitening components that the production of melanin is reduced by only inhibiting the activity of the tyrosinase, the whitening mechanisms of the lotus extract and the Glycyrrhiza glabra extract in the “whitening factor” can inhibit pigmentation by inhibiting synthesis of the melanin, inhibiting transfer of melanin globule, resisting oxidation and improving skin microcirculation, so as to achieve the effect of special whitening. Meanwhile, reduction in melanin can cause decrease in hydrature of skin so as to give rise to a series problems such as drying, while the Dendrobium candidum polysaccharide has many biological activities beneficial to a human body; through the synergistic synergy of the three components, the compounded complexes can reduce synthesis of the melanin of skin's melanophores, enhance the ability of the Dendrobium candidum polysaccharide to promote expression of keratinocyte hydration related factors, inhibit expression of inflammation related factors, promote the ability of lipid synthesis and then exert multiple effects of whitening, enhancement in moisturizing of skin, alleviation in inflammation and discomfort of skin and improvement in barrier function of the epidermis. In addition, the complexes are safe, reliable and effective, and may be applied in the preparation of skin care products or skin formulations.

### BRIEF DESCRIPTION OF DRAWINGS

FIG. 1 is a diagram showing effects of polysaccharide-whitening factor complexes A1-A5 on a content of melanin in melanocytes.

FIG. 2 is a diagram showing effects of polysaccharide-whitening factor complexes B1-B5 on a content of melanin in melanocytes.

FIG. 3 is a diagram showing effects of polysaccharide-whitening factor complexes A1-A5 on the expression of aquaporin AQP3.

FIG. 4 is a diagram showing effects of polysaccharide-whitening factor complexes B1-B5 on the expression of aquaporin AQP3.

FIG. 5 is a diagram showing effects of polysaccharide-whitening factor complexes A1-A5 on the production of ROS induced by UVB.

FIG. 6 is a diagram showing effects of polysaccharide-whitening factor complexes B1-B5 on the production of ROS induced by UVB.

FIG. 7A is a diagram showing effects of polysaccharide-whitening factor complexes A1-A5 on the inflammatory factors IL-1α; FIG. 7B is a diagram showing effects of polysaccharide-whitening factor complexes A1-A5 on the inflammatory factors IL-1β; FIG. 7C is a diagram showing effects of polysaccharide-whitening factor complexes A1-A5 on the inflammatory factors IL-6; and FIG. 7D is a diagram showing effects of polysaccharide-whitening factor complexes A1-A5 on the inflammatory factors TNF-α.

FIG. 8A is a diagram showing effects of polysaccharide-whitening factor complexes B1-B5 on the inflammatory factors IL-1α; FIG. 8B is a diagram showing effects of polysaccharide-whitening factor complexes B1-B5 on the inflammatory factors IL-1β; FIG. 8C is a diagram showing effects of polysaccharide-whitening factor complexes B1-B5 on the inflammatory factors IL-6; and FIG. 8D is a diagram showing effects of polysaccharide-whitening factor complexes B1-B5 on the inflammatory factors TNF-α.

FIG. 9A is a diagram showing effects of polysaccharide-whitening factor complexes A1-A5 on the expression of a Ca²⁺-induced lipid synthesis factor CerS3; FIG. 9B is a diagram showing effects of polysaccharide-whitening factor complexes A1-A5 on the expression of a Ca²⁺-induced lipid synthesis factor CerS4; FIG. 9C is a diagram showing effects of polysaccharide-whitening factor complexes A1-A5 on the expression of a Ca²⁺-induced lipid synthesis factor FAS; FIG. 9D is a diagram showing effects of polysaccharide-whitening factor complexes A1-A5 on the expression of a Ca²⁺-induced lipid synthesis factor SPT; FIG. 9E is a diagram showing effects of polysaccharide-whitening factor complexes A1-A5 on the expression of a Ca²⁺-induced lipid synthesis factor ELOVL1; FIG. 9F is a diagram showing effects of polysaccharide-whitening factor complexes A1-A5 on the expression of a Ca²⁺-induced lipid synthesis factor ELOVL4; and FIG. 9G is a diagram showing effects of polysaccharide-whitening factor complexes A1-A5 on the expression of a Ca²⁺-induced lipid synthesis factor ELOVL6.

FIG. 10A is a diagram showing effects of polysaccharide-whitening factor complexes B1-B5 on the expression of a Ca²⁺-induced lipid synthesis factor CerS3; FIG. 10B is a diagram showing effects of polysaccharide-whitening factor complexes B1-B5 on the expression of a Ca²⁺-induced lipid synthesis factor CerS4; FIG. 10C is a diagram showing effects of polysaccharide-whitening factor complexes B1-B5 on the expression of a Ca²⁺-induced lipid synthesis factor FAS; FIG. 10D is a diagram showing effects of polysaccharide-whitening factor complexes B1-B5 on the expression of a Ca²⁺-induced lipid synthesis factor SPT; FIG. 10E is a diagram showing effects of polysaccharide-whitening factor complexes B1-B5 on the expression of a Ca²⁺-induced lipid synthesis factor ELOVL1; FIG. 10F is a diagram showing effects of polysaccharide-whitening factor complexes B1-B5 on the expression of a Ca²⁺-induced lipid synthesis factor ELOVL4; and FIG. 10G is a diagram showing effects of polysaccharide-whitening factor complexes B1-B5 on the expression of a Ca²⁺-induced lipid synthesis factor ELOVL6.

FIG. 11 is a diagram showing effects of polysaccharide-whitening factor complexes A1-A5 on a content of intracellular lipid.

FIG. 12 is a diagram showing effects of polysaccharide-whitening factor complexes B1-B5 on a content of intracellular lipid.

### DETAILED DESCRIPTION

Next, the concept and technical effects of the present disclosure will be clearly and completely described in combination with embodiments to sufficiently understand the objectives, features and effects of the present disclosure. Obviously, the described embodiments are only some embodiments of the present disclosure, but not all the embodiments. Based on the embodiments of the present disclosure, other embodiments obtained by those skilled in the art without creative efforts fall into the protection scope of the present disclosure.

The lotus extract: the lotus extract is purchased from Ashland Group in the United States with a production batch number of 001684RA00, and the extraction process is consistent with the extraction technology described in the US patent document U.S. Pat. No. 8,668,939B2.

### Example 1

Preparation of Dendrobium candidum Polysaccharide
(1) 1 kg of Dendrobium candidum was crushed into crude particles and extracted twice with water, wherein a first solid-liquid ratio (g/mL) was 1:40, and the extraction was conducted for 2 h at 100° C., and a second solid-liquid ratio (g/mL) was 1:10, and the extraction was conducted for 1.5 h at 100° C. The extracting solutions were respectively filtrated by 100 mesh, and the obtained filtrate were combined for tubular centrifugation. The centrifuged extracting solution was concentrated to 1/10 volume of the filtrate at 70-80° C.; absolute ethanol was added to the concentrated solution to adjust a concentration of ethanol to 80%; alcohol precipitation was conducted for 2 h; 300-mesh filtration was performed to collect precipitates; and then the ethanol was evaporated.
(2) The precipitates were added into water for re-dissolution (a concentration was 0.5%) to obtain a polysaccharide solution; the polysaccharide solution was evenly mixed with a sevage reagent [chloroform:n-butanol=4:1 (v:v)] in a ratio of 5:1 (v:v); the above mixed solution was sufficiently shaken for 20 min for standing until obvious layering appeared; the sevage in the lower layer and proteins in the middle layer were released, and such operations were repeated for about 10 times; and the polysaccharide solution was collected for later use.
(3) First, macroporous resin AB-8 was activated by washing with distilled water, then soaking with 3-5% HCl solution and 3-5% NaOH solution for 24 h in sequence; the activated macroporous resin AB-8 was washed with the distilled water to be neutral; then the washing solution was removed by suction filtration. An appropriate amount of activated macroporous resin was added in to the polysaccharide solution, placed in a shaker for shaking for 4 h, and subjected to suction filtration to obtain a polysaccharide solution without pigment. The polysaccharide solution was collected, concentrated to 1/10 volume of the filtrate at 60-70° C. and underwent freeze drying for 48 h at −80° C. to obtain polysaccharide freeze-dried powder.
(4) The polysaccharide freeze-dried powder (20 mg) was weighed and dissolved into 1 mL of distilled water; and a sample was centrifuged for 10 min at 6000 r/min after being completely dissolved into water. The supernatant was taken and loaded. The sample was slowly added to a G100 sephadex column with a dropper, wherein the sample must be slowly added dropwise along the wall of the chromatographic column so as to avoid the situation that uneven gel surface is generated to affect the separation effect. The sample was eluted by using the distilled water as an eluent at a flow rate of 1 mL·min⁻¹, the eluted solution was collected in 6 min/tube, 50 μL of solution was taken from each tube until the loading solution was completely collected; the loading solution was concentrated to 1/10 volume of filtrate at 60-70° C.; and then the concentrated solution was subjected to freeze drying for 48 h at −80□ to obtain the Dendrobium candidum polysaccharide.

Detection on Molecular Weight of Dendrobium candidum Polysaccharide
(1) 10 mg of the Dendrobium candidum polysaccharide sample prepared in the above steps were precisely weighed and dissolved into 10 mL of 0.1 M NaNO₃ aqueous solution to prepare an aqueous solution with a final concentration of 1 mg/mL, and the obtained aqueous solution was filtrated through a filter with a pore size of 0.22 μm for later use. (2) The filtered Dendrobium candidum polysaccharide solution sample was analyzed by a gel chromatography-differential-multi-angle laser light scattering system, with a injection volume of 100 μL, where the chromatographic conditions were as follows: gel size exclusion chromatography columns Ohpak SB-805 HQ (300×8 m), Ohpak SB-804 HQ (300×8 mm), Ohpak SB-803 HQ (300×8 mm) connected in series, a column temperature of 45° C., a mobile phase A (0.1M NaNO₃), a flow rate of 0.4 ml/min, an elution gradient of isocratic elution for 100 min. By detection, the molecular weight of the Dendrobium candidum polysaccharide was shown as 0.3-27 kDa.

The preparation of the Glycyrrhiza glabra extract may be seen in Chinese patent CN108176079A in details.

Preparation of a Dendrobium candidum polysaccharide aqueous solution: every 15 mg of dried Dendrobium candidum polysaccharide obtained was dissolved into 1 mL of sterilized deionized water, and the obtained solution was filtered through a 0.22 μm microporous membrane to prepare a 15 mg/mL Dendrobium candidum polysaccharide aqueous solution.

Preparation of a lotus extract aqueous solution: every 15 mg of lotus extract obtained was dissolved into 1 mL of sterilized deionized water, and the obtained solution was filtered through a 0.22 μm microporous membrane to prepare a 15 mg/mL lotus extract aqueous solution.

Preparation of a Glycyrrhiza glabra extract aqueous solution: every 15 mg of dried Glycyrrhiza glabra extract obtained was dissolved into 1 mL of sterilized deionized water, and the obtained solution was filtered through a 0.22 μm microporous membrane to obtain a 15 mg/mL Glycyrrhiza glabra extract aqueous solution.

### Example 2

Groups:

Any treatment was not conducted for a blank group.

UVB irradiation was conducted only in an ultraviolet irradiator for a UVB group.

Only 10 μL of CaCl₂ aqueous solution (with a concentration of 2 mol/L) was added for a Ca²⁺ group.

Only 2 μL of 15 mg/mL Dendrobium candidum polysaccharide aqueous solution was added for administration for a Dendrobium candidum alone group.

Only 2 μL of 15 mg/mL lotus extract aqueous solution was added for administration for a lotus extract alone group.

Only 2 μL of 15 mg/mL Glycyrrhiza glabra aqueous solution was added for administration for a Glycyrrhiza glabra alone group.

The complexes A1, A2, A3, A4 and A5 were prepared by simply mixing the following three components in the following mass ratios. Dendrobium candidum polysaccharide:lotus extract: Glycyrrhiza glabra extract=1:1:1 (Complex A1), 2:2:3 (Complex A2), 2:3:2 (Complex A3), 3:2:2 (Complex A4), and 5:1:1 (Complex A5).

Complex A1 group: 5 mg of dried Dendrobium candidum polysaccharide, 5 mg of lotus extract and 5 mg of Glycyrrhiza glabra extract were weighted, mixed, dissolved into 1 mL of sterilized deionized water, and filtered via a 0.22 μm microporous membrane to obtain a mixture aqueous solution. 2 μL of mixture aqueous solution was taken for administration.

Complex A2 group: 4 mg of dried Dendrobium candidum polysaccharide, 4 mg of lotus extract and 6 mg of Glycyrrhiza glabra extract were weighted, mixed, dissolved into 1 mL of sterilized deionized water, and filtered via a 0.22 μm microporous membrane to obtain a mixture aqueous solution. 2 μL of mixture aqueous solution was taken for administration.

Complex A3 group: 4 mg of dried Dendrobium candidum polysaccharide, 6 mg of lotus extract and 4 mg of Glycyrrhiza glabra extract were weighted, mixed, dissolved into 1 mL of sterilized deionized water, and filtered via a 0.22 μm microporous membrane to obtain a mixture aqueous solution. 2 μL of mixture aqueous solution was taken for administration.

Complex A4 group: 6 mg of dried Dendrobium candidum polysaccharide, 4 mg of lotus extract and 4 mg of Glycyrrhiza glabra extract were weighted, mixed, dissolved into 1 mL of sterilized deionized water, and filtered via a 0.22 μm microporous membrane to obtain a mixture aqueous solution. 2 μL of mixture aqueous solution was taken for administration.

Complex A5 group: 10 mg of dried Dendrobium candidum polysaccharide, 2 mg of lotus extract and 2 mg of Glycyrrhiza glabra extract were weighted, mixed, dissolved into 1 mL of sterilized deionized water, and filtered via a 0.22 μm microporous membrane to obtain a mixture aqueous solution. 2 μL of mixture aqueous solution was taken for administration.

The complexes B1, B2, B3, B4 and B5 were prepared by simply mixing the following three components in the following mass ratios. Dendrobium candidum polysaccharide:lotus extract: Glycyrrhiza glabra extract=3:2:1 (Complex B1), 3:1:2 (Complex B2), 3:2:2 (Complex B3), 3:3:2 (Complex B4), and 3:2:3 (Complex B5).

Complex B1 group: 7.5 mg of dried Dendrobium candidum polysaccharide, 5 mg of lotus extract and 2.5 mg of Glycyrrhiza glabra extract were weighted, mixed, dissolved into 1 mL of sterilized deionized water, and filtered via a 0.22 μm microporous membrane to obtain a mixture aqueous solution. 2 μL of mixture aqueous solution was taken for administration.

Complex B2 group: 7.5 mg of dried Dendrobium candidum polysaccharide, 2.5 mg of lotus extract and 5 mg of Glycyrrhiza glabra extract were weighted, mixed, dissolved into 1 mL of sterilized deionized water, and filtered via a 0.22 μm microporous membrane to obtain a mixture aqueous solution. 2 μL of mixture aqueous solution was taken for administration.

Complex B3 group: 6 mg of dried Dendrobium candidum polysaccharide, 4 mg of lotus extract and 4 mg of Glycyrrhiza glabra extract were weighted, mixed, dissolved into 1 mL of sterilized deionized water, filtered via a 0.22 μm microporous membrane to obtain a mixture aqueous solution. 2 μL of mixture aqueous solution was taken for administration.

Complex B4 group: 5.25 mg of dried Dendrobium candidum polysaccharide, 5.25 mg of lotus extract and 3.5 mg of Glycyrrhiza glabra extract were weighted, mixed, dissolved into 1 mL of sterilized deionized water, and filtered via a 0.22 μm microporous membrane to obtain a mixture aqueous solution. 2 μL of mixture aqueous solution was taken for administration.

Complex B5 group: 5.25 mg of dried Dendrobium candidum polysaccharide, 3.5 mg of lotus extract and 5.25 mg of Glycyrrhiza glabra extract were weighted, mixed, dissolved into 1 mL of sterilized deionized water, and filtered via a 0.22 μm microporous membrane to obtain a mixture aqueous solution. 2 μL of mixture aqueous solution was taken for administration.

### Effect Example

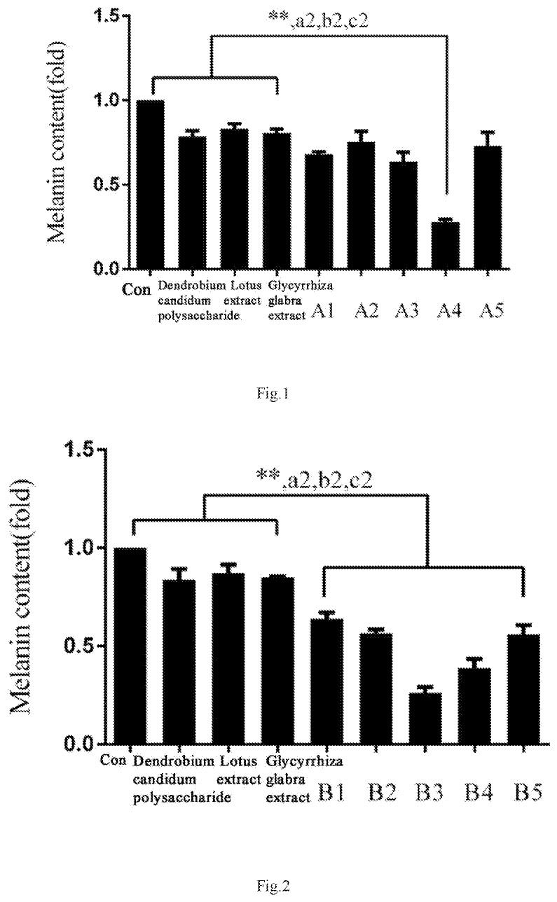

(1) Determination on Content of Melanin in Cells
A) A375 cells were inoculated in a 6-well plate at 1×10⁵ cells/well, and 2 mL of DMEM culture medium (containing 10% fetal bovine serum) was added to each well for culture. After incubation for 12 h in a CO₂ incubator, a Dendrobium candidum polysaccharide aqueous solution, a lotus extract aqueous solution, a Glycyrrhiza glabra extract aqueous solution and polysaccharide-whitening factor complexes A1-A5 were respectively added according to groups in Example 2, and an untreated group was used as a cell control group (blank group). After incubation for 48 h, the culture medium was discarded, the cells were washed once with PBS. 100 μL of cell lysate was added to each well for lysis on ice for 30 min; then the lysed product was centrifuged for 15 min at 12000 r/min; the supernatant was discarded; 150 μL of 1 M NaOH solution containing 10% DMSO was added to precipitates; the obtained mixture underwent water bath for 30 min at 80□; the supernatant was collected and added to a 96-well plate; and an absorbance value (OD value) was detected at a wavelength of 475 nm with a microplate reader to serve as a detection index of the melanin content. Melanin content=(OD value of assay well-OD value of blank control)/(OD value of cell control group-OD value of blank control). The results were shown in Table 1 and FIG. 1.

TABLE 1
Effects of polysaccharide-whitening factor complexes A1-A5
on the content of melanin in melanocytes (relative value)

| Group | Melanin |
|---|---|
| Blank | 1 |
| Dendrobium candidum polysaccharide | 0.7867 ± 0.06* |
| Lotus extract | 0.8333 ± 0.05 |
| Glycyrrhiza glabra extract | 0.8067 ± 0.04* |
| A1 | 0.6800 ± 0.03** |
| A2 | 0.7533 ± 0.11** |
| A3 | 0.6367 ± 0.10**^{,b1} |
| A4 | 0.2800 ± 0.03**^{,a2,b2,c2} |
| A5 | 0.7300 ± 0.14** |

Note:
compared with the blank group, *P < 0.05, **P < 0.01. Compared with the dendrobium candidum polysaccharide alone group, a2 P < 0.01. Compared with the lotus extract alone group, b1 P < 0.05, b2 P < 0.01. Compared with glycyrrhiza glabra extract alone group, c2 P < 0.01.

It can be seen that compared with the blank control group, all of the polysaccharide-whitening factor complexes A1-A5 can significantly reduce the content of intracellular melanin, indicating that the polysaccharide-whitening factore complexes A1-A5 have a good whitening effect. The Dendrobium candidum polysaccharide alone group and the Glycyrrhiza glabra extract alone group can reduce the content of the melanin in the melanocytes, and have a whitening effect. Where, the polysaccharide-whitening factor complex A3 is significantly better than the lotus extract in the effect of reducing the content of the melanin, and the polysaccharide-whitening factor complex A4 (Dendrobium candidum polysaccharide:lotus extract: Glycyrrhiza glabra extract=3:2:2) is significantly better than the Dendrobium candidum polysaccharide alone group, the lotus extract alone group and the Glycyrrhiza glabra extract alone group in the effect of reducing the content of the melanin, indicating that compared with the Dendrobium candidum polysaccharide, the lotus extract or the Glycyrrhiza glabra extract, the polysaccharide-whitening factor complex A4 (Dendrobium candidum polysaccharide:lotus extract: Glycyrrhiza glabra extract=3:2:2) has a better whitening effect. The polysaccharide-whitening factor complex A4 (Dendrobium candidum polysaccharide:lotus extract: Glycyrrhiza glabra extract=3:2:2) has the best whitening effect.
B) The effects of the polysaccharide-whitening factor complexes B1-B5 on the content of the melanin in the melanocytes were detected according to the above-mentioned specific embodiment.

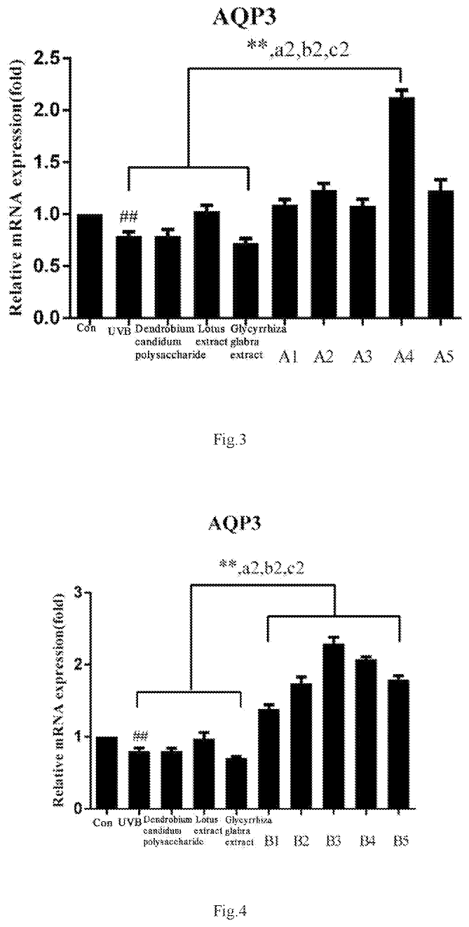

The results were shown in Table 2 and FIG. 2.

TABLE 2
Effects of polysaccharide-whitening factor complexes B1-B5
on the content of melanin in melanocytes (relative value)

| Group | Melanin |
|---|---|
| Blank | 1 |
| Dendrobium candidum polysaccharide | 0.8400 ± 0.09* |
| Lotus extract | 0.8733 ± 0.07 |
| Glycyrrhiza glabra extract | 0.8500 ± 0.01* |
| B1 | 0.6400 ± 0.06**^{,a2,b2,c2} |
| B2 | 0.5667 ± 0.03**^{,a2,b2,c2} |
| B3 | 0.2633 ± 0.05**^{,a2,b2,c2} |
| B4 | 0.3900 ± 0.08**^{,a2,b2,c2} |
| B5 | 0.5600 ± 0.08**^{,a2,b2,c2} |

Note:
compared with blank group, *P < 0.05, **P < 0.01. Compared with the dendrobium candidum polysaccharide alone group, a2 P < 0.01. Compared with the lotus extract alone group, b2 P < 0.01. Compared with the glycyrrhiza glabra extract alone group, c2 P < 0.01.

It can be seen that compared with the blank control group, all of the polysaccharide-whitening factor complexes B1-B5 can significantly reduce the content of intracellular melanin, indicating that the polysaccharide-whitening factor complexes B1-B5 have good whitening effect. The Dendrobium candidum polysaccharide alone group and the Glycyrrhiza glabra extract alone group can reduce the content of the melanin in the melanocytes, and have the whitening effect. Where, the polysaccharide-whitening factor complexes B1-B5 are significantly better than the Dendrobium candidum polysaccharide alone group, the lotus extract alone group and the Glycyrrhiza glabra extract alone group in the effect of reducing the content of intracellular melanin, indicating that compared with the Dendrobium candidum polysaccharide, the lotus extract and the Glycyrrhiza glabra extract, the polysaccharide-whitening factor complexes B1-B5 have a better whitening effect. Among them, the polysaccharide-whitening factor complex B3 (Dendrobium candidum polysaccharide:lotus extract: Glycyrrhiza glabra extract=3:2:2) has the strongest effect of reducing the content of the intracellular melanin and has the best whitening effect.

(2) Detection of the Expression of HaCaT Cell Hydration Related mRNA Via Real-Time PCR
A) HaCaT cells were inoculated to a 6-well plate at 2×10⁵ cells/well, and 2 mL of DMEM culture medium (containing 10% fetal bovine serum) was added to each well for culture. After incubation for 12 h in a CO₂ incubator, a Dendrobium candidum polysaccharide aqueous solution, a lotus extract aqueous solution, a Glycyrrhiza glabra extract aqueous solution and polysaccharide-whitening factor complexes A1-A5 were respectively added according to groups in Example 2. After treatment for 24 h, the untreated group was used as cell control group (blank group). Except for the blank group, the culture plate was placed in an ultraviolet irradiator for UVB (20 mJ) irradiation and then incubated for 2 h in an incubator, and the cells were lysed for extraction of total RNA. The expression level of mRNA in the HaCaT cell hydration related gene AQP3 was detected via Real-Time PCR to serve as a detection index for hydration. The results were shown in Table 3 and FIG. 3.

TABLE 3
Effects of polysaccharide-whitening factor complexes A1-A5
on the expression of aquaporin AQP3 (relative value)

| Group | AQP3 |
|---|---|
| Blank | 1 |
| UVB | 0.79 ± 0.04^{##} |
| Dendrobium candidum polysaccharide | 0.79 ± 0.06 |
| Lotus extract | 1.03 ± 0.06** |
| Glycyrrhiza glabra extract | 0.72 ± 0.04 |
| A1 | 1.09 ± 0.05^{a2,c2} |
| A2 | 1.24 ± 0.06**^{,a2,b2,c2} |
| A3 | 1.08 ± 0.07**^{,a2,c2} |
| A4 | 2.13 ± 0.07**^{,a2,b2,c2} |
| A5 | 1.23 ± 0.10**^{,a2,b2,c2} |

Note:
by comparing UVB group with blank group, ^{##}P < 0.01; and compared with UVB group, **P < 0.01. By comparing the polysaccharide-whitening factor complexes with the dendrobium candidum polysaccharide alone group, a2 P < 0.01. By comparing the polysaccharide-whitening factor complexes with the lotus extract alone group, b2 P < 0.01. By comparing the polysaccharide-whitening factor complexes with the glycyrrhiza glabra extract alone group, c2P < 0.01.

It can be seen that compared with the blank control group, the UVB group can significantly reduce the expression level of AQP3, resulting in a drying problem. Compared with the UVB group, the lotus extract alone group can significantly up-regulate the expression level of AQP3, and the polysaccharide-whitening factor complexes A2-A5 can significantly up-regulate the expression the of AQP3, so as to play the moisturizing effect. The effects of polysaccharide-extracting whitening factor complexes A1-A5 in up-regulating the expression level of AQP3 are significantly higher than those of the Dendrobium candidum polysaccharide alone group and the Glycyrrhiza glabra alone group. Compared with the lotus extract alone group, the effects of the polysaccharide-whitening factor complexes A2, A4 and A5 in up-regulating the expression level of AQP3 are significantly higher than those of lotus extract. The above data show that compared with the Dendrobium candidum polysaccharide, the lotus extract or the Glycyrrhiza glabra extract, the polysaccharide-whitening factor complexes A2, A3 and A5 have a better moisturizing effect. Where, the polysaccharide-whitening factor complex A4 (Dendrobium candidum polysaccharide:lotus extract: Glycyrrhiza glabra extract=3:2:2) has the strongest effect of up-regulating the expression level of AQP3, indicating that polysaccharide-whitening factor complex A4 (Dendrobium candidum polysaccharide:lotus extract: Glycyrrhiza glabra extract=3:2:2) has the best moisturizing effect.
B) The effects of the polysaccharide-whitening factor complexes B1-B5 on the expression of aquaporin AQP3 were detected according to the above-mentioned specific embodiment.

The results were shown in Table 4 and FIG. 4.

TABLE 4
Effects of polysaccharide-whitening factor complexes B1-B5
on the expression of aquaporin AQP3 (relative value)

| Group | AQP3 |
|---|---|
| Blank | 1 |
| UVB | 0.80 ± 0.05^{##} |
| Dendrobium candidum polysaccharide | 0.80 ± 0.04 |
| Lotus extract | 0.97 ± 0.09* |
| Glycyrrhiza glabra extract | 0.71 ± 0.02 |
| B1 | 1.38 ± 0.06**^{,a2,b2,c2} |
| B2 | 1.75 ± 0.09**^{,a2,b2,c2} |
| B3 | 2.30 ± 0.09**^{,a2,b2,c2} |
| B4 | 2.08 ± 0.03**^{,a2,b2,c2} |
| B5 | 1.79 ± 0.06**^{,a2,b2,c2} |

Note:
by comparing the UVB group with the blank group, ^{##}P < 0.01; compared with the UVB group, *P < 0.05, **P < 0.01. By comparing the polysaccharide-whitening factor complexes with the dendrobium candidum polysaccharide alone group, a2 P < 0.01. By comparing the polysaccharide-whitening factor complexes with the lotus extract alone group, b2 P < 0.01. By comparing the polysaccharide-whitening factor complexes with the glycyrrhiza glabra extract alone group, c2 P < 0.01.

It can be seen that compared with the blank control group, the UVB group can significantly reduce the expression level of AQP3, resulting in a drying problem. Compared with the UVB group, the lotus extract alone group can up-regulate the expression level of AQP3, and has the moisturizing effect. The polysaccharide-whitening factor complexes B1-B5 can significantly up-regulate the expression level of AQP3, and have the moisturizing effect. The effects of polysaccharide-whitening factor complexes B1-B5 in up-regulating the expression level of AQP3 are significantly higher than those of the Dendrobium candidum polysaccharide alone group, the lotus extract alone group or the Glycyrrhiza glabra alone group, indicating that compared with the Dendrobium candidum polysaccharide, the lotus extract or the Glycyrrhiza glabra extract, the polysaccharide-whitening factor complexes B1-B5 have a better moisturizing effect. Where, the polysaccharide-whitening factor complex B3 (Dendrobium candidum polysaccharide:lotus extract: Glycyrrhiza glabra extract=3:2:2) has the strongest effect of promoting the expression of AQP3, indicating that it has the best moisturizing effect.

(3) Detection of the Yield of Reactive Oxygen Species in HaCaT Cells Via Flow Cytometry
A) HaCaT cells were inoculated into a 6-well plate in 2×10⁵ cells/well, and 2 mL of DMEM culture medium (containing 10% fetal bovine serum) was added to each well for culture. After incubation for 24 h in a CO₂ incubator, a Dendrobium candidum polysaccharide aqueous solution, a lotus extract aqueous solution, a Glycyrrhiza glabra extract aqueous solution and polysaccharide-whitening factor complexes A1-A5 were respectively added according to groups in Example 2. After treatment for 24 h, the untreated group was used as a cell control group (blank group). Except for the blank group, the culture plate was placed in an ultraviolet irradiator for UVB (1J) irradiation, a DCFH-DA fluorescent probe diluted with a serum-free culture medium was added, and then the culture plate was placed in the incubator for incubation for 20 min to load the cells. The cell suspension was collected, and the yield of reactive oxygen species (ROS) in the HaCaT cells was detected by the flow cytometry to serve as a detection index for oxidative stress.

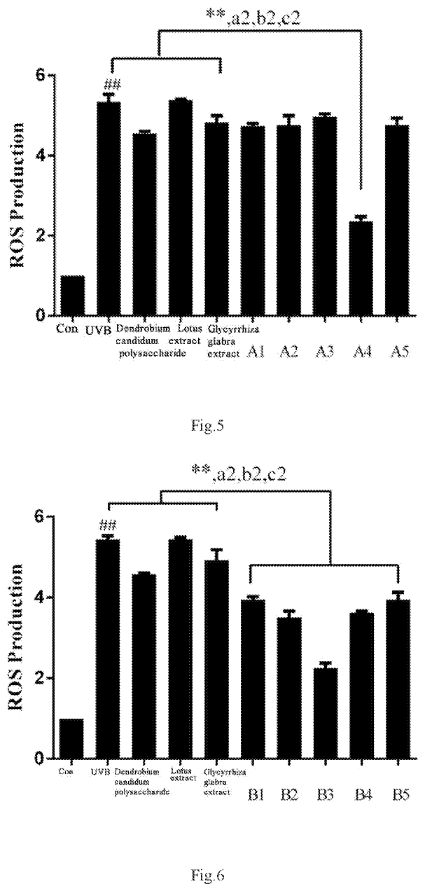

The results were shown in Table 5 and FIG. 5.

TABLE 5
Effects of polysaccharide-whitening factor complexes A1-A5
on the production of ROS induced by UVB

| Group | ROS |
|---|---|
| Blank | 1 |
| UVB | 5.34 ± 0.19^{##} |
| Dendrobium candidum polysaccharide | 4.56 ± 0.05** |
| Lotus extract | 5.40 ± 0.02 |
| Glycyrrhiza glabra extract | 4.82 ± 0.17** |
| A1 | 4.74 ± 0.06**^{,b2} |
| A2 | 4.75 ± 0.24**^{,b2} |
| A3 | 4.97 ± 0.08*^{,b1} |
| A4 | 2.36 ± 0.12**^{,a2,b2,c2} |
| A5 | 4.76 ± 0.17**^{,b2} |

Note:
by comparing the UVB group with the blank group, ^{##}P < 0.01; compared with the UVB group, *P < 0.05, **P < 0.01. By comparing the polysaccharide-whitening factor complexes with the dendrobium candidum polysaccharide alone group, a2 P < 0.01. By comparing the polysaccharide-whitening factor complexes with the lotus extract alone group, b1 P < 0.05, and b2 P < 0.01. By the comparing polysaccharide-whitening factor complexes with the glycyrrhiza glabra extract alone group, c2 P < 0.01.

It can be seen that compared with the blank control group, the yield of ROS in the UVB group is significantly increased, indicating that UVB induces excessive production of the ROS, and oxidative damages are caused. Compared with the UVB group, the Dendrobium candidum polysaccharide alone group and the Glycyrrhiza glabra extract alone group can both reduce the production of ROS caused by the UVB, and exert the effect of resisting the oxidative damages, and all the polysaccharide-whitening factor complexes A1-A5 can reduce the production of ROS and exert the effect of resisting the oxidative damages. Compared with the Dendrobium candidum polysaccharide alone group, the lotus extract alone group and the Glycyrrhiza glabra extract alone group, the effect of the polysaccharide-whitening factor complex A4 in inhibiting the production of ROS induced by the UVB is significantly stronger than those of the Dendrobium candidum polysaccharide, the lotus extract and the Glycyrrhiza glabra extract, indicating that compared with the Dendrobium candidum polysaccharide, the lotus extract or the Glycyrrhiza glabra extract, the polysaccharide-whitening factor complex A4 (Dendrobium candidum polysaccharide:lotus extract: Glycyrrhiza glabra extract=3:2:2) has better antioxidant effect.
B) The effects of the polysaccharide-whitening factor complexes B1-B5 on the production of ROS induced by the UVB were detected according to the above specific embodiments.

The results were shown in Table 6 and FIG. 6.

TABLE 6
Effects of polysaccharide-whitening factor complexes B1-B5
on the production of ROS induced by UVB

| Group | ROS |
|---|---|
| Blank | 1 |
| UVB | 5.44 ± 0.09^{##} |
| Dendrobium candidum polysaccharide | 4.58 ± 0.03** |
| Lotus extract | 5.44 ± 0.05 |
| Glycyrrhiza glabra extract | 4.92 ± 0.26** |
| B1 | 3.95 ± 0.08**^{, a2,b2,c2} |
| B2 | 3.50 ± 0.08**^{, a2,b2,c2} |
| B3 | 2.26 ± 0.12**^{, a2,b2,c2} |
| B4 | 3.62 ± 0.04**^{, a2,b2,c2} |
| B5 | 3.94 ± 0.19**^{, a2,b2,c2} |

Note:
by comparing the UVB group with the blank group, ^{##}P < 0.01; compared with the UVB group, *P < 0.01, **P < 0.01. By comparing the polysaccharide-whitening factor complexes with the dendrobium candidum polysaccharide alone group, a2 P < 0.01. By comparing the polysaccharide-whitening factor complexes with the lotus extract alone group, b2 P < 0.01. By comparing the polysaccharide-whitening factor complexes with the glycyrrhiza glabra extract alone group, c2 P < 0.01.

It can be seen that compared with the blank control group, the production of ROS in the UVB group is significantly increased, indicating that the UVB induces excessive production of the ROS, and oxidative damages are caused. Compared with the UVB group, the Dendrobium candidum polysaccharide alone group and the Glycyrrhiza glabra extract alone group can both reduce the production of ROS caused by the UVB, and exert the effect of resisting the oxidative damages, and all the polysaccharide-whitening factor complexes B1-B5 can reduce the production of ROS, and exert the effect of resisting the oxidative damages. Compared with the Dendrobium candidum polysaccharide alone group, the lotus extract alone group and the Glycyrrhiza glabra extract alone group, the effects of the polysaccharide-whitening factor complexes B1-B5 in reducing the production of ROS are significantly stronger than those of the Dendrobium candidum polysaccharide, the lotus extract and the Glycyrrhiza glabra extract, indicating that compared with the Dendrobium candidum polysaccharide, the lotus extract or the Glycyrrhiza glabra extract, the polysaccharide-whitening factor complexes B1-B5 have better antioxidant effect. Among them, the polysaccharide-whitening factor complex B3 (Dendrobium candidum polysaccharide:lotus extract: Glycyrrhiza glabra extract=3:2:2) has the strongest effect of inhibiting the production of ROS, indicating that it has the best antioxidant effect.

(4) Detection on the Expression of HaCaT Cell Inflammation Related mRNA Via Real-Time PCR
A) HaCaT cells were inoculated into a 6-well plate in 2×10⁵ cells/well, and 2 mL of DMEM culture medium (containing 10% fetal bovine serum) was added to each well for culture. After incubation for 24 h in a CO₂ incubator, a Dendrobium candidum polysaccharide aqueous solution, a lotus extract aqueous solution, a Glycyrrhiza glabra extract aqueous solution and the polysaccharide-whitening factor complexes A1-A5 were respectively added according to groups in Example 2. After treatment for 24 h, the untreated group was used as cell control group (blank group). Except for the blank group, the culture plate was placed in the ultraviolet irradiator for UVB (15 mJ) irradiation, and the cells were lysed for extraction of total RNA. The expression level of mRNA of HaCaT cell inflammation related factors (IL-1α, IL-1β, IL-6 and TNF-α) was detected via a Real-Time PCR experiment to serve as a detection index for an inflammatory response.

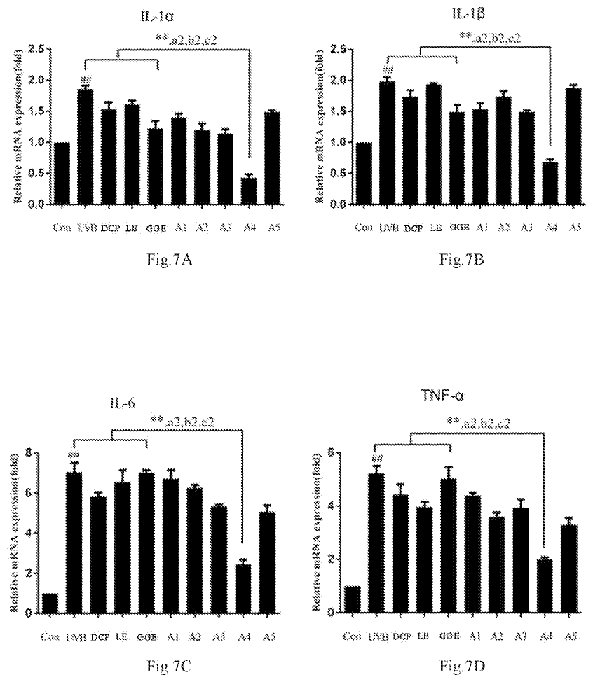

The results were seen in Table 7, FIG. 7A, FIG. 7B, FIG. 7C, and FIG. 7D.

TABLE 7
Effects of polysaccharide-whitening factor complexes A1-A5 on UVB-induced
inflammatory response

| Group | IL-1α | IL-1β | IL-6 | TNF-α |
|---|---|---|---|---|
| Blank | 1 | 1 | 1 | 1 |
| UVB | 1.86 ± 0.06^{##} | 1.98 ± 0.06^{##} | 7.05 ± 0.46^{##} | 5.22 ± 0.28^{##} |
| Dendrobium | 1.53 ± 0.11** | 1.73 ± 0.11** | 5.84 ± 0.19** | 4.43 ± 0.38** |
| candidum | | | | |
| polysaccharide | | | | |
| (DCP) | | | | |
| Lotus extract | 1.61 ± 0.06** | 1.94 ± 0.02 | 6.54 ± 0.62 | 3.96 ± 0.20** |
| (LE) | | | | |
| Glycyrrhiza | 1.22 ± 0.12** | 1.49 ± 0.11** | 7.03 ± 0.11 | 5.03 ± 0.44 |
| glabra extract | | | | |
| (GGE) | | | | |
| A1 | 1.40 ± 0.06**^{,b1} | 1.54 ± 0.10**^{,a1,b2} | 6.72 ± 0.43 | 4.41 ± 0.08**^{,c1} |
| A2 | 1.20 ± 0.10**^{, a2,b2} | 1.73 ± 0.09**^{,b1} | 6.26 ± 0.15^{*,c1} | 3.60 ± 0.16**^{, a2,c2} |
| A3 | 1.14 ± 0.07**^{, a2,b2} | 1.49 ± 0.03**^{,a2,b2} | 5.35 ± 0.09^{**,b2,c2} | 3.94 ± 0.31**^{,c2} |
| A4 | 0.42 ± 0.06**^{, a2,b2,c2} | 0.69 ± 0.04**^{,a2,b2,c2} | 2.44 ± 0.24^{**,a2,b2,c2} | 1.99 ± 0.08**^{,a2,b2,c2} |
| A5 | 1.48 ± 0.03** | 1.87 ± 0.05** | 5.08 ± 0.32^{**,b2,c2} | 3.30 ± 0.26**^{,a2,b1,c2} |

Note:
by comparing the UVB group with blank group, ^{##}P < 0.01; compared with the UVB group, *P < 0.01, **P < 0.01. By comparing the polysaccharide-whitening factor complexes with the dendrobium candidum polysaccharide alone group, a1 P < 0.05, and a2 P < 0.01. By comparing the polysaccharide-whitening factor complexes with the lotus extract alone group, b1 P < 0.05, and b2 P < 0.01. By comparing the polysaccharide-whitening factor complexes with the glycyrrhiza glabra extract alone group, c1 P < 0.01, and c2 P < 0.01.

It can be seen that compared with the blank control group, the expression levels of inflammatory factors in the UVB group are significantly increased, indicating that UVB irradiation can lead to cellular inflammatory responses. Compared with the UVB group, the Dendrobium candidum polysaccharide alone group can significantly reduce the levels of inflammatory factors IL-1α, IL-1β, IL-6 and TNF-α and exert the anti-inflammatory effect, and the lotus extract and the Glycyrrhiza glabra extract can significantly reduce the levels of some inflammatory factors and exert the partial anti-inflammatory effect. The polysaccharide-extracting whitening factor complex A1 can significantly reduce the levels of some inflammatory factors, and the polysaccharide-extracting whitening factor complexes A2-A5 can significantly reduce the levels of inflammatory factors IL-1α, IL-1β, IL-6 and TNF-α and exert good anti-inflammatory effect. Compared with the Dendrobium candidum polysaccharide alone group, the lotus extract alone group and the Glycyrrhiza glabra extract alone group, the effects of the polysaccharide-whitening factor complexes A1, A2, A3 and A5 in reducing the expression level of some inflammatory factors are partially stronger than that of the Dendrobium candidum polysaccharide, the lotus extract or the Glycyrrhiza glabra extract. The polysaccharide-whitening factor complex A4 can reduce the expression levels of all inflammatory factors and has the effect stronger than that of the Dendrobium candidum polysaccharide, the lotus extract or the Glycyrrhiza glabra extract, indicating that compared with the Dendrobium candidum polysaccharide, the lotus extract or the Glycyrrhiza glabra extract, the polysaccharide-whitening factor complex A4 (Dendrobium candidum polysaccharide:lotus extract: Glycyrrhiza glabra extract=3:2:2) has the best anti-inflammatory effect.
B) The effects of the polysaccharide-whitening factor complexes B1-B5 on inflammatory response caused by UVB were detected according to the above specific embodiment.

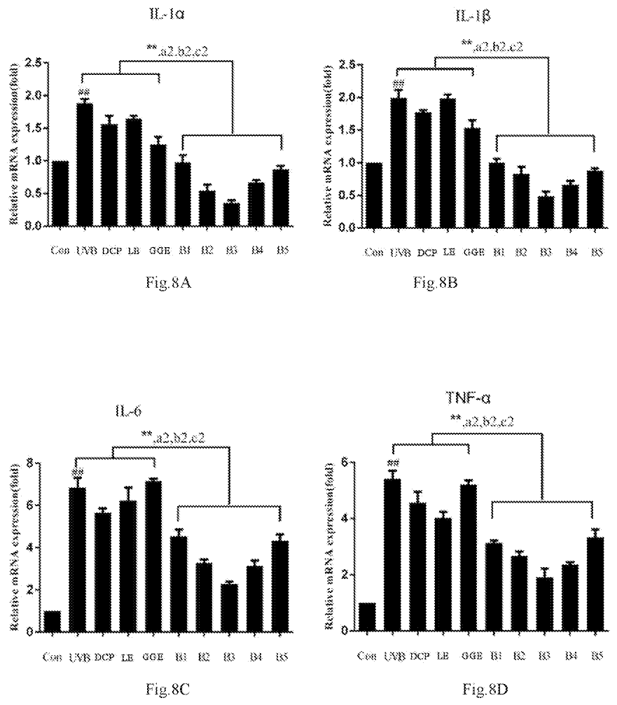

The results were shown in Table 8, FIG. 8A, FIG. 8B, FIG. 8C, and FIG. 8D.

TABLE 8
Effects of polysaccharide-whitening factor complexes B1-B5 on UVB-induced
inflammatory response

| Group | IL-1α | IL-1β | IL-6 | TNF-α |
|---|---|---|---|---|
| Blank | 1 | 1 | 1 | 1 |
| UVB | 1.89 ± 0.06^{##} | 2.00 ± 0.12^{##} | 6.85 ± 0.46^{##} | 5.42 ± 0.28^{##} |
| Dendrobium | 1.56 ± 0.13** | 1.77 ± 0.03* | 5.67 ± 0.19** | 4.57 ± 0.38** |
| candidum | | | | |
| polysaccharide | | | | |
| (DCP) | | | | |
| Lotus extract | 1.65 ± 0.05** | 1.99 ± 0.06 | 6.24 ± 0.62 | 4.03 ± 0.20** |
| (LE) | | | | |
| Glycyrrhiza | 1.25 ± 0.12** | 1.53 ± 0.12** | 7.16 ± 0.11 | 5.20 ± 0.16 |
| glabra extract | | | | |
| (GGE) | | | | |
| B1 | 0.98 ± 0.11**^{,a2,b2,c2} | 1.00 ± 0.06**^{,a2,b2,c2} | 4.55 ± 0.31**^{,a2,b2,c2} | 3.14 ± 0.08**^{,a2,b2,c2} |
| B2 | 0.54 ± 0.09**^{,a2,b2,c2} | 0.83 ± 0.10**^{,a2,b2,c2} | 3.29 ± 0.15**^{,a2,b2,c2} | 2.67 ± 0.16**^{,a2,b2,c2} |
| B3 | 0.36 ± 0.04**^{,a2,b2,c2} | 0.49 ± 0.07**^{,a2,b2,c2} | 2.29 ± 0.09**^{,a2,b2,c2} | 1.91 ± 0.31**^{,a2,b2,c2} |
| B4 | 0.67 ± 0.04**^{,a2,b2,c2} | 0.66 ± 0.06**^{,a2,b2,c2} | 3.15 ± 0.25**^{,a2,b2,c2} | 2.36 ± 0.08**^{,a2,b2,c2} |
| B5 | 0.87 ± 0.05**^{,a2,b2,c2} | 0.88 ± 0.03**^{,a2,b2,c2} | 4.32 ± 0.32**^{,a2,b2,c2} | 3.34 ± 0.27**^{,a2,b2,c2} |

Note:
by comparing the UVB group with the blank group, ^{##}P < 0.01; compared with the UVB group, *P < 0.05, and ** P < 0.01. By comparing the polysaccharide-whitening factor complexes with the dendrobium candidum polysaccharide alone group, a2 P < 0.01. By comparing the polysaccharide-whitening factor complexes with the lotus extract alone group, b2 P < 0.01. By comparing the polysaccharide-whitening factor complexes with the glycyrrhiza glabra extract alone group, c2 P < 0.01.

It can be seen that compared with the blank control group, the expression levels of inflammatory factors in the UVB group are significantly increased, indicating that UVB irradiation may lead to cellular inflammatory responses. Compared with the UVB group, the Dendrobium candidum polysaccharide alone group can significantly reduce the levels of the inflammatory factors IL-1α, IL-1β, IL-6, TNF-α, and exert the anti-inflammatory effect; and the lotus extract and the Glycyrrhiza glabra extract can significantly reduce the levels of some inflammatory factors and exert the partial anti-inflammatory effect. All the polysaccharide-polysaccharide complexes B1-B5 can significantly reduce the expression levels of the inflammatory factors IL-1α, IL-1β, IL-6 and TNF-α, indicating that the polysaccharide-whitening factor complexes B1-B5 have good anti-inflammatory effect. In addition, compared with the Dendrobium candidum polysaccharide alone group, the lotus extract alone group and the Glycyrrhiza glabra extract alone group, the effects of the polysaccharide-whitening factor complexes B1-B5 in reducing the expression levels of all inflammatory factors are stronger than that of the Dendrobium candidum polysaccharide, the lotus extract or the Glycyrrhiza glabra extract, indicating that compared with the Dendrobium candidum polysaccharide, the lotus extract or the Glycyrrhiza glabra extract, the polysaccharide-whitening factor complexes B1-B5 have better anti-inflammatory effects. Among them, the polysaccharide-whitening factor complex B3 (Dendrobium candidum polysaccharide:lotus extract: Glycyrrhiza glabra extract=3:2:2) has the strongest effect of reducing the expression level of inflammatory factors, indicating that it has the best anti-inflammatory effect.

(5) Detection on the Expression of HaCaT Cell Lipid Synthesis Related mRNA Via Real-Time PCR

HaCaT cells were inoculated into a 6-well plate in 2×10⁵ cells/well, and 2 mL of DMEM culture medium (containing 10% fetal bovine serum) was added to each well for culture. After incubation for 24 h in a CO₂ incubator, a Dendrobium candidum polysaccharide aqueous solution, a lotus extract aqueous solution, a Glycyrrhiza glabra extract aqueous solution and the polysaccharide-whitening factor complexes A1-A5 were respectively added according to groups in Example 2. The untreated group was used as the cell control group (blank group). Except for the blank group, 10 mM Ca²⁺ was added to each group. After treatment for 48 h, the cells were lysed for extraction of total RNA. The expression level of mRNA of HaCaT cell lipid synthesis related factors (CerS3, CerS4, FAS, SPT, ELOVL1, ELOVL4 and ELOVL6) were detected via a Real-Time PCR experiment to serve as an index for detecting a lipid content.

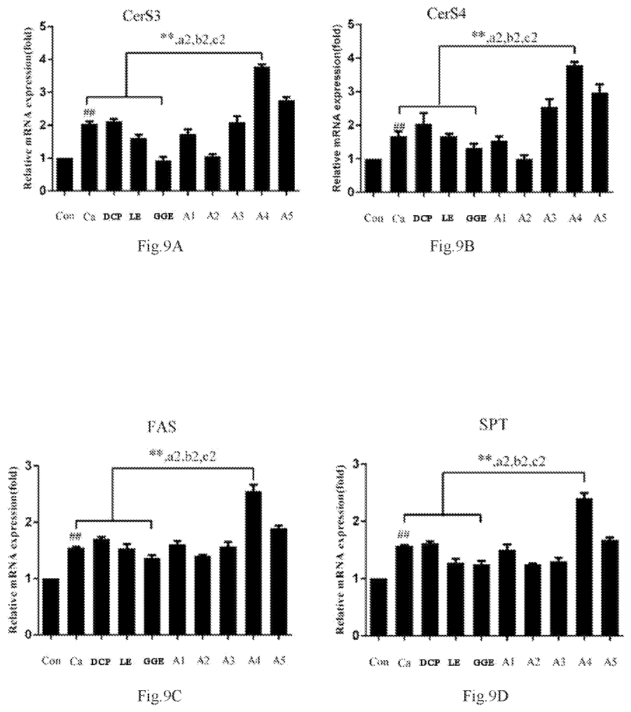

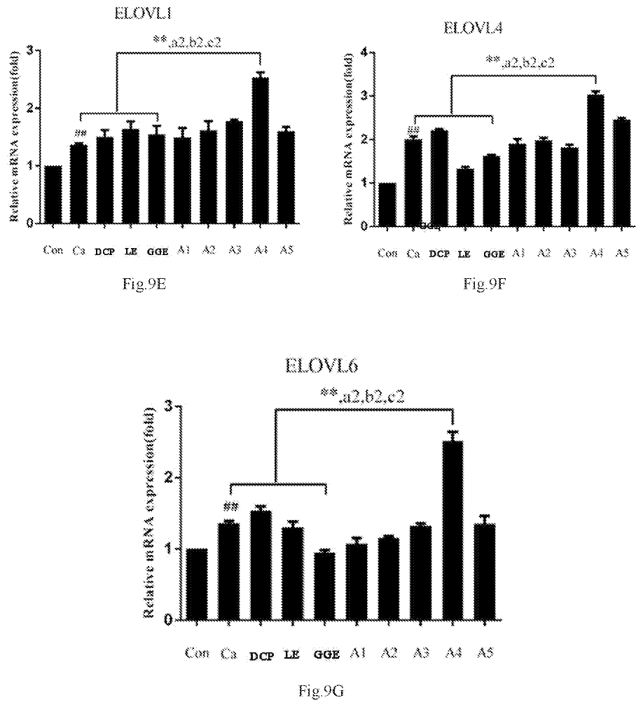

The results were shown in Table 9, FIG. 9A, FIG. 9B, FIG. 9C, FIG. 9D, FIG. 9E, FIG. 9F, and FIG. 9G.

TABLE 9
Effects of polysaccharide-whitening factor complexes A1-A5 on Ca²⁺-induced
lipid synthesis factor expression

| Group | Cers3 | CerS4 | FAS |
|---|---|---|---|
| Blank | 1 | 1 | 1 |
| Ca²⁺ | 2.04 ± 0.08^{##} | 1.68 ± 0.13^{##} | 1.54 ± 0.02^{##} |
| Dendrobium candidum polysaccharide | 2.12 ± 0.08 | 2.05 ± 0.32 | 1.70 ± 0.03* |
| (DCP) | | | |
| Lotus extract (LE) | 1.62 ± 0.10 | 1.67 ± 0.08 | 1.55 ± 0.08 |
| Glycyrrhiza glabra extract (GGE) | 0.93 ± 0.10 | 1.32 ± 0.13 | 1.37 ± 0.05 |
| A1 | 1.73 ± 0.14^{,c2} | 1.55 ± 0.12 | 1.61 ± 0.05^{c2} |
| A2 | 1.06 ± 0.07 | 1.00 ± 0.11 | 1.41 ± 0.02 |
| A3 | 2.10 ± 0.18^{,b2,c2} | 2.55 ± 0.23**^{,a1,b2,c2} | 1.57 ± 0.08^{c1} |
| A4 | 3.79 ± 0.06**^{, a2,b2,c2} | 3.79 ± 0.10**^{,a2,b2,c2} | 2.56 ± 0.11**^{,a2,b2,c2} |
| A5 | 2.77 ± 0.10**^{,a2,b2,c2} | 2.98 ± 0.24**^{,a2,b2,c2} | 1.90 ± 0.05**^{,a1,b2,c2} |
| SPT | ELOVL1 | ELOVL4 | ELOVL6 |
| 1 | 1 | 1 | 1 |
| 1.57 ± 0.02^{##} | 1.37 ± 0.02^{##} | 2.01 ± 0.06^{##} | 1.36 ± 0.03^{##} |
| 1.62 ± 0.03 | 1.50 ± 0.12 | 2.21 ± 0.03** | 1.54 ± 0.06* |
| 1.28 ± 0.07 | 1.65 ± 0.12 | 1.33 ± 0.05 | 1.30 ± 0.08 |
| 1.25 ± 0.06 | 1.55 ± 0.15 | 1.62 ± 0.02 | 0.95 ± 0.03 |
| 1.51 ± 0.09^{b2,c2} | 1.49 ± 0.16 | 1.91 ± 0.10^{b2,c2} | 1.07 ± 0.08 |
| 1.25 ± 0.02 | 1.62 ± 0.16 | 1.98 ± 0.06^{b2,c2} | 1.16 ± 0.02^{c1} |
| 1.31 ± 0.05 | 1.78 ± 0.02** | 1.82 ± 0.06^{b2,c2} | 1.32 ± 0.04^{c2} |
| 2.41 ± 0.09**^{,a2,b2,c2} | 2.53 ± 0.09**^{,a2,b2,c2} | 3.04 ± 0.07^{**,a2,b2,c2} | 2.52 ± 0.13**^{,a2,b2,c2} |
| 1.68 ± 0.04^{b2,c2} | 1.60 ± 0.08 | 2.45 ± 0.04^{**,a2,b2,c2} | 1.35 ± 0.11^{c2} |

Note:
by comparing the Ca²⁺ group with the blank group, ^{##}P < 0.01; compared with the Ca²⁺ group, *P < 0.05, and **P < 0.01. By comparing the polysaccharide-whitening factor complexes with the dendrobium candidum polysaccharide alone group, a1 P < 0.05, and a2 P < 0.01. By comparing the polysaccharide-whitening factor complexes with the lotus extract alone group, b2 P < 0.01. By comparing the polysaccharide-whitening factor complexes with the glycyrrhiza glabra extract alone group, c1 P < 0.05, and c2 P < 0.01.

It can be seen that compared with the blank control group, the expression levels of the lipid synthesis related factors CerS3, CerS4, FAS, SPT, ELOVL1, ELOVL4 and ELOVL6 in the Ca²⁺ group are all significantly increased, indicating that Ca²⁺ stimulation may induce lipid synthesis. Compared with the Ca²⁺ group, the Dendrobium candidum polysaccharide alone group can promote increase in expression levels of Ca²⁺-induced FAS, ELOVL4 and ELOVL6, indicating that the Dendrobium candidum polysaccharide alone group can partially promote lipid synthesis, and the polysaccharide-whitening factor complex A3 can increase the expression level of some lipid synthesis related genes CerS4 and ELOVL1, and the polysaccharide-whitening factor complex A5 can increase the expression level of some lipid synthesis related genes CerS3, CerS4, FAS and ELOVL4, indicating that the polysaccharide-whitening factor complexes A3 and A5 can partially promote lipid synthesis. The polysaccharide-whitening factor complex A4 can promote the expression of all the lipid synthesis related factors CerS3, CerS4, FAS, ELOVL4 and ELOVL6, indicating that the polysaccharide-whitening factor complex A4 has the effect of completely promoting lipid synthesis. Compared with the Dendrobium candidum polysaccharide alone group, the effect of the polysaccharide-extracting whitening factor complex A3 in increasing the expression level of the lipid synthesis related gene CerS4 is better than that of the Dendrobium candidum polysaccharide, and the effect of the polysaccharide-extracting whitening factor complex A5 in increasing the expression levels of the lipid synthesis related factors CerS3, CerS4, FAS and ELOVL4 was better than that of the Dendrobium candidum polysaccharide, and the effect of the polysaccharide-whitening factor complex A4 in increasing the expression levels of all the lipid synthesis related factors CerS3, CerS4, FAS, SPT, ELOVL1, ELOVL4 and ELOVL6 is significantly better than that of the Dendrobium candidum polysaccharide, indicating that compared with the Dendrobium candidum polysaccharide, the lotus extract or the Glycyrrhiza glabra extract, the polysaccharide-whitening factor complex A4 (Dendrobium candidum polysaccharide:lotus extract: Glycyrrhiza glabra extract=3:2:2) has the best effect of promoting lipid synthesis.
B) The effects of polysaccharide-whitening factor complexes B1-B5 on the expression levels of Ca²⁺-induced lipid synthesis factors were detected according to the above-mentioned specific embodiment.

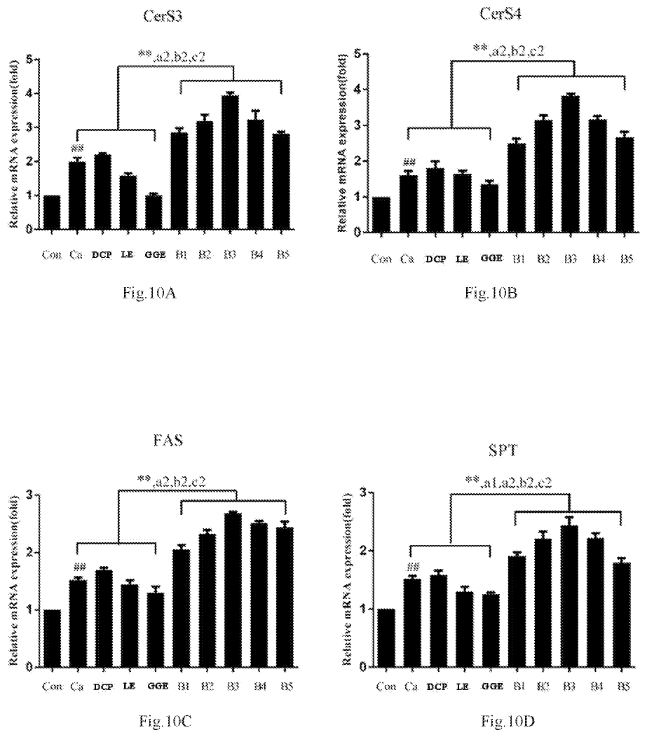

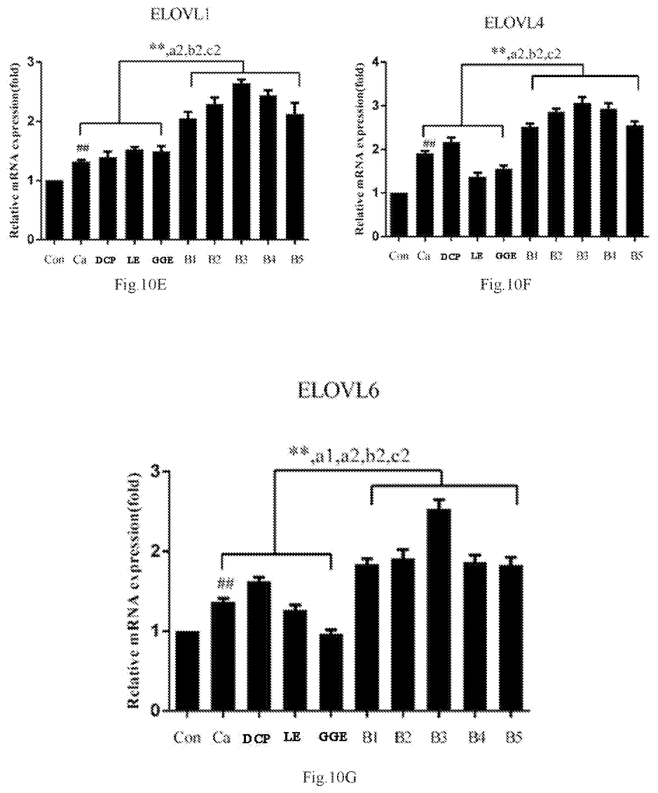

The results were shown in Table 10, FIG. 10A, FIG. 10B, FIG. 10C, FIG. 10D, FIG. 10E, FIG. 10F, and FIG. 10G.

TABLE 10
Effects of polysaccharide-whitening factor complexes B1-B5 on the expression
levels of Ca²⁺-induced lipid synthesis factors

| Group | CerS3 | CerS4 | FAS |
|---|---|---|---|
| Blank | 1 | 1 | 1 |
| Ca²⁺ | 1.99 ± 0.08^{##} | 1.60 ± 0.12^{##} | 1.52 ± 0.05^{##} |
| Dendrobium candidum polysaccharide | 2.21 ± 0.03 | 1.82 ± 0.18 | 1.70 ± 0.04* |
| (DCP) | | | |
| Lotus extract (LE) | 1.58 ± 0.06 | 1.65 ± 0.08 | 1.44 ± 0.07 |
| Glycyrrhiza glabra extract (GGE) | 1.00 ± 0.05 | 1.35 ± 0.10 | 1.30 ± 0.11 |
| B1 | 2.86 ± 0.13**^{,a2,b2,c2} | 2.50 ± 0.13**^{,a2,b2,c2} | 2.05 ± 0.08**^{,a2,b2,c2} |
| B2 | 3.19 ± 0.19**^{,a2,b2,c2} | 3.15 ± 0.14**^{,a2,b2,c2} | 2.32 ± 0.07**^{,a2,b2,c2} |
| B3 | 3.95 ± 0.08**^{,a2,b2,c2} | 3.83 ± 0.06**^{,a2,b2,c2} | 2.69 ± 0.02**^{,a2,b2,c2} |
| B4 | 3.24 ± 0.25**^{,a2,b2,c2} | 3.16 ± 0.10**^{,a2,b2,c2} | 2.51 ± 0.04**^{,a2,b2,c2} |
| B5 | 2.82 ± 0.06**^{,a2,b2,c2} | 2.67 ± 0.15**^{,a2,b2,c2} | 2.44 ± 0.09**^{,a2,b2,c2} |
| SPT | ELOVL1 | ELOVL4 | ELOVL6 |
| 1 | 1 | 1 | 1 |
| 1.52 ± 0.05^{##} | 1.32 ± 0.03^{##} | 1.91 ± 0.05^{##} | 1.37 ± 0.04^{##} |
| 1.59 ± 0.08 | 1.40 ± 0.09 | 2.17 ± 0.10* | 1.62 ± 0.06** |
| 1.30 ± 0.08 | 1.53 ± 0.04 | 1.37 ± 0.09 | 1.27 ± 0.06 |
| 1.25 ± 0.04 | 1.50 ± 0.09 | 1.56 ± 0.07 | 0.97 ± 0.05 |
| 1.92 ± 0.06**^{,a2,b2,c2} | 2.05 ± 0.11 | 2.51 ± 0.08**^{,a2,b2,c2} | 1.84 ± 0.07**^{,a1,b2,c2} |
| 2.21 ± 0.12**^{,a2,b2,c2} | 2.29 ± 0.11**^{,a2,b2,c2} | 2.86 ± 0.07**^{,a2,b2,c2} | 1.91 ± 0.12**^{,a2,b2,c2} |
| 2.44 ± 0.14**^{,a2,b2,c2} | 2.64 ± 0.07**^{,a2,b2,c2} | 3.07 ± 0.12**^{,a2,b2,c2} | 2.54 ± 0.11**^{,a2,b2,c2} |
| 2.22 ± 0.08**^{,a2,b2,c2} | 2.44 ± 0.08**^{,a2,b2,c2} | 2.93 ± 0.13**^{,a2,b2,c2} | 1.87 ± 0.09**^{,a1,b2,c2} |
| 1.80 ± 0.08**^{,a1,b2,c2} | 2.12 ± 0.19**^{,a2,b2,c2} | 2.55 ± 0.09**^{,a2,b2,c2} | 1.83 ± 0.10**^{,a1,b2,c2} |

Note:
by comparing the Ca²⁺ group with the blank group, ^{##}P < 0.01; compared with the Ca²⁺ group, *P < 0.05, and **P < 0.01. By comparing the polysaccharide-whitening factor complexes with the dendrobium candidum polysaccharide alone group, a1 P < 0.05, and a2 P < 0.01. By comparing the polysaccharide-whitening factor complexes with the lotus extract alone group, b2 P < 0.01. By comparing the polysaccharide-whitening factor complexes with the glycyrrhiza glabra extract alone group, c2 P < 0.01.

It can be seen that compared with the blank control group, the expression levels of the lipid synthesis related factors CerS3, CerS4, FAS, SPT, ELOVL1, ELOVL4 and ELOVL6 in the Ca²⁺ group are significantly increased, indicating that Ca²⁺ stimulation may induce lipid synthesis. Compared with the Ca²⁺ group, the Dendrobium candidum polysaccharide alone group can promote increase in expression levels of Ca²⁺-induced FAS, ELOVL4 and ELOVL6, indicating that Dendrobium candidum polysaccharide alone group can partially promote lipid synthesis. The polysaccharide-whitening factor complexes B1-B5 can promote the expression of all the lipid synthesis related factors CerS3, CerS4, FAS, SPT, ELOVL1, ELOVL4 and ELOVL6, indicating that the polysaccharide-whitening factor complexes B1-B5 have the effect of comprehensively promoting lipid synthesis. Compared with the Dendrobium candidum polysaccharide alone group, the effects of the polysaccharide-whitening factor complexes B1-B5 in increasing the expression levels of all the lipid synthesis related factors are significantly higher than that of the Dendrobium candidum polysaccharide, indicating that compared with the Dendrobium candidum polysaccharide, the lotus extract or the Glycyrrhiza glabra extract, the polysaccharide-whitening factor complexes B1-B5 have better effect of promoting lipid synthesis. Among them, the polysaccharide-whitening factor complex B3 (Dendrobium candidum polysaccharide:lotus extract: Glycyrrhiza glabra extract=3:2:2) has the strongest effect of promoting lipid synthesis, indicating that it has the best effect of promoting lipid synthesis.

(6) Detection on the Content of Lipid in HaCaT Cells Via Fluorescence Staining

HaCaT cells were inoculated into a 6-well plate in 2×10⁵ cells/well, and 2 mL of DMEM culture medium (containing 10% fetal bovine serum) was added to each well for culture. After incubation for 24 h in a CO₂ incubator, a Dendrobium candidum polysaccharide aqueous solution, a lotus extract aqueous solution, a Glycyrrhiza glabra extract aqueous solution and polysaccharide-whitening factor complexes A1-A5 were respectively added according to groups in Example 2. The untreated group was used as the cell control group (blank group). Except for the blank group, 10 mM Ca²⁺ was added to each group. After treatment for 48 h, a Hoechst dye with an original concentration of 1.8 mM was diluted with a complete culture medium to obtain a 20 μM Hoechst working solution. Nile red dye powder was dissolved into ethanol to obtain a 2 mg/mL Nile red solution, and 10 μL of Nile red solution was added to each 4 mL of PBS to prepare a working solution. A Hoechst nucleus dye was added to cells and stained for 15 min in an incubator, then 0.6-1 mL of Nile red staining solution was added, the plate was placed in the incubator for culture for 20 min to label lipid components, and the fluorescence intensity was observed under a fluorescent inverted microscope to serve as an auxiliary detection index for a lipid content.

TABLE 11
Effects of polysaccharide-whitening factor complexes
A1-A5 on intracellular lipid content

| Group | Lipid content |
|---|---|
| Blank | 1 |
| Ca²⁺ | 1.20 ± 0.05^{##} |
| Dendrobium candidum polysaccharide | 1.15 ± 0.05 |
| Lotus extract | 0.89 ± 0.06 |
| Glycyrrhiza glabra extract | 0.82 ± 0.01 |
| A1 | 1.22 ± 0.06^{b2,c2} |
| A2 | 0.98 ± 0.06^{c1} |
| A3 | 1.12 ± 0.07^{b2,c2} |
| A4 | 1.79 ± 0.10**^{,a2,b2,c2} |
| A5 | 1.32 ± 0.06^{a1,b2,c2} |

Note:
by comparing the Ca²⁺ group with the blank group, ^{##}P < 0.01; and compared with the Ca²⁺ group, **P < 0.01. By comparing the polysaccharide-whitening factor complexes with the dendrobium candidum polysaccharide alone group, a1 P < 0.05, and a2 P < 0.01. By comparing the polysaccharide-whitening factor complexes with the lotus extract alone group, b2 P < 0.01. By comparing the polysaccharide-whitening factor complexes with the glycyrrhiza glabra extract alone group, c1 P < 0.05, and c2 P < 0.01.

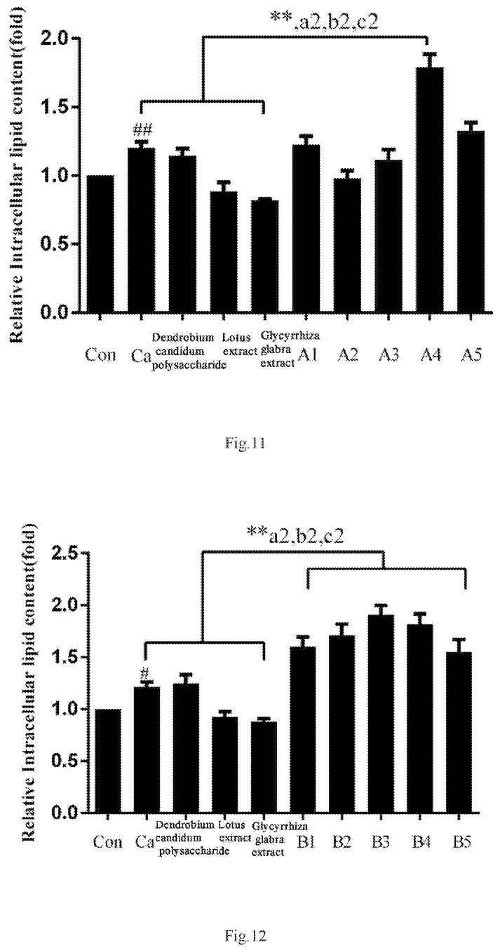

The results were shown in Table 11 and FIG. 11.

It can be seen that compared with the blank group, the lipid content of the Ca²⁺ group is significantly increased, indicating that Ca²⁺ stimulation may induce lipid synthesis. Compared with the Ca²⁺ group, treatment with the polysaccharide-whitening factor complex A4 can promote lipid synthesis induced by Ca²⁺ stimulation, indicating that the polysaccharide-whitening factor complex A4 has the effect of promoting lipid synthesis. Compared with the Dendrobium candidum polysaccharide alone group, the polysaccharide-whitening factor complexes A4 and A5 have a stronger effect of promoting lipid synthesis. Compared with the lotus extract alone group, the polysaccharide-whitening factor complexes A1, A3, A4, and A5 have a stronger effect of promoting lipid synthesis. Compared with the Glycyrrhiza glabra extract, the polysaccharide-whitening factor complexes A1-A5 have a stronger effect of promoting lipid synthesis. The above results show that the ability of the polysaccharide-whitening factor complex A4 to promote the increase of lipid is significantly better than that of the Dendrobium candidum polysaccharide, the lotus extract or the Glycyrrhiza glabra extract, the polysaccharide-whitening factor complex A4 (Dendrobium candidum polysaccharide:lotus extract: Glycyrrhiza glabra extract=3:2:2) has the best effect of promoting lipid synthesis.
B) The effects of the polysaccharide-whitening factor complexes B1-B5 on a content of intracellular lipid were detected according to the above specific embodiment.

The results were shown in Table 12 and FIG. 12.

TABLE 12
Effects of polysaccharide-whitening factor complexes B1-B5
on the content of intracellular lipid

| Group | Lipid content |
|---|---|
| Blank | 1 |
| Ca²⁺ | 1.21 ± 0.05^{#} |
| Dendrobium candidum polysaccharide | 1.25 ± 0.09 |
| Lotus extract | 0.93 ± 0.05 |
| Glycyrrhiza glabra extract | 0.88 ± 0.03 |
| B1 | 1.60 ± 0.09**^{,a2,b2,c2} |
| B2 | 1.71 ± 0.11**^{,a2,b2,c2} |
| B3 | 1.91 ± 0.09**^{,a2,b2,c2} |
| B4 | 1.82 ± 0.10**^{,a2,b2,c2} |
| B5 | 1.55 ± 0.12**^{,a2,b2,c2} |

Note:
by comparing the Ca²⁺ group with blank group, ^{##}P < 0.05; and compared with the Ca²⁺ group, **P < 0.01. By comparing the polysaccharide-whitening factor complexes with the dendrobium candidum polysaccharide alone group, a2 P < 0.01. By comparing the polysaccharide-whitening factor complexes with the lotus extract alone group, b2 P < 0.01. By comparing the polysaccharide-whitening factor complexes with the glycyrrhiza glabra extract alone group, c2 P < 0.01.

It can be seen that compared with the blank group, the lipid content of the Ca²⁺ group is significantly increased, indicating that Ca²⁺ stimulation may induce lipid synthesis. Compared with the Ca²⁺ group, treatment with the polysaccharide-whitening factor complexes B1-B5 can promote the lipid synthesis induced by Ca²⁺ stimulation, indicating that the polysaccharide-whitening factor complexes B1-B5 have the effect of promoting lipid synthesis. Compared with the Dendrobium candidum polysaccharide alone group, the lotus extract alone group and the Glycyrrhiza glabra extract alone group, the polysaccharide-whitening factor complexes B1-B5 have a stronger effect of promoting lipid synthesis, indicating that compared with the Dendrobium candidum polysaccharide, the lotus extract or the Glycyrrhiza glabra extract, the polysaccharide-whitening factor complexes B1-B5 have a better effect of promoting lipid synthesis. Among them, the polysaccharide-whitening factor complex B3 (Dendrobium candidum polysaccharide:lotus extract: Glycyrrhiza glabra extract=3:2:2) has the best effect of promoting lipid synthesis, indicating that it has the best effect of promoting lipid synthesis.

The present disclosure has been illustrated in detail by the above-mentioned specific embodiments, but the present disclosure is not limited to the above-mentioned embodiments, various variations can also be made within the knowledge scope possessed by persons of ordinary skill in the art and without departing from the spirit of the present disclosure. In addition, embodiments of the present disclosure and features in embodiments can be combined with each other without conflict.

1. A composition, comprising Dendrobium candidum polysaccharide, a lotus extract and a Glycyrrhiza glabra extract.

2. The composition according to claim 1, wherein a mass ratio of the Dendrobium candidum polysaccharide to the lotus extract to the Glycyrrhiza glabra extract is (1-5):(1-3):(1-3).

3. The composition according to claim 1, wherein the mass ratio of the Dendrobium candidum polysaccharide to the lotus extract to the Glycyrrhiza glabra extract is (1-3):(1-3):(1-3).

4. The composition according to claim 1, wherein the mass ratio of the Dendrobium candidum polysaccharide to the lotus extract to the Glycyrrhiza glabra extract is 3:(1-3):(1-3).

5. The composition according to claim 1, wherein the Dendrobium candidum polysaccharide, the lotus extract and the Glycyrrhiza glabra extract are extracted from Dendrobium candidum, lotus and Glycyrrhiza glabra, as raw materials, respectively.

6. A product comprising the composition according to claim 1.

7. A product comprising the composition according to claim 2.

8. A product comprising the composition according to claim 3.

9. A product comprising the composition according to claim 4.

10. A product comprising the composition according to claim 5.

11. The product according to claim 6, wherein the product has at least one function of (a)-(j):
(a) reducing a content of melanin;
(b) promoting expression of aquaporin AQP3;
(c) inhibiting production of reactive oxygen species (ROS);
(d) inhibiting expression of inflammatory factors;
(e) promoting expression of lipid synthesis-related factors;
(f) promoting lipid synthesis;
(g) whitening;
(h) moisturizing;
(i) resisting oxidation; and
(j) resisting inflammation.

12. The product according to claim 7, wherein the product has at least one function of (a)-(j):
(a) reducing a content of melanin;
(b) promoting expression of aquaporin AQP3;
(c) inhibiting production of reactive oxygen species (ROS);
(d) inhibiting expression of inflammatory factors;
(e) promoting expression of lipid synthesis-related factors;
(f) promoting lipid synthesis;
(g) whitening;
(h) moisturizing;
(i) resisting oxidation; and
(j) resisting inflammation.

13. The product according to claim 8, wherein the product has at least one function of (a)-(j):
(a) reducing a content of melanin;
(b) promoting expression of aquaporin AQP3;
(c) inhibiting production of reactive oxygen species (ROS);
(d) inhibiting expression of inflammatory factors;
(e) promoting expression of lipid synthesis-related factors;
(f) promoting lipid synthesis;
(g) whitening;
(h) moisturizing;
(i) resisting oxidation; and
(j) resisting inflammation.

14. The product according to claim 9, wherein the product has at least one function of (a)-(j):
(a) reducing a content of melanin;
(b) promoting expression of aquaporin AQP3;
(c) inhibiting production of reactive oxygen species (ROS);
(d) inhibiting expression of inflammatory factors;
(e) promoting expression of lipid synthesis-related factors;
(f) promoting lipid synthesis;
(g) whitening;
(h) moisturizing;
(i) resisting oxidation; and
(j) resisting inflammation.

15. The product according to claim 10, wherein the product has at least one function of (a)-(j):
(a) reducing a content of melanin;
(b) promoting expression of aquaporin AQP3;
(c) inhibiting production of reactive oxygen species (ROS);
(d) inhibiting expression of inflammatory factors;
(e) promoting expression of lipid synthesis-related factors;
(f) promoting lipid synthesis;
(g) whitening;
(h) moisturizing;
(i) resisting oxidation; and
(j) resisting inflammation.

16. The product according to claim 6, wherein the product is a cosmetic or a drug.

17. The product according to claim 7, wherein the product is a cosmetic or a drug.

18. The product according to claim 8, wherein the product is a cosmetic or a drug.

19. The product according to claim 9, wherein the product is a cosmetic or a drug.

20. The product according to claim 16, wherein the cosmetic further comprises a excipient, wherein the excipient is at least one selected from the group consisting of a preservative, a chelating agent, a flavor, a moisturizer, a colorant, an emulsifier, an antioxidant and a skin conditioner.
